(19) United States
(12) Patent Application Publication
(10) Pub. No.: US 2025/0058324 A1
(43) Pub. Date: Feb. 20, 2025

(54) AUTOMATED MOLECULAR OPERATING SYSTEM

(71) Applicant: GENE TARGET TECHNOLOGY CO., LTD., Kaohsiung (TW)

(72) Inventors: Yi-Fang CHEN, Kaohsiung (TW); Suz-Kai HSIUNG, Kaohsiung (TW); Chun-Wei HUANG, Kaohsiung (TW); Yin-Lin LI, Kaohsiung (TW); Yu-Ying WU, Kaohsiung (TW)

(21) Appl. No.: 18/512,897

(22) Filed: Nov. 17, 2023

(30) Foreign Application Priority Data
Aug. 18, 2023 (TW) 112131138

(51) Int. Cl. B01L 3/00

(52) U.S. Cl. CPC B01L 3/50857; B01L 3/50851; B01L 2200/0647; B01L 2200/0673; B01L 2200/16; B01L 2300/0838; B01L 2400/043

(57) ABSTRACT

An automated molecular operating system includes at least one centrifuge tube carrying module, a transport module, a plurality of temperature control modules, a capping module, a magnetic field module and an automated processing module. The automated processing module is electrically connected to the transport module, the temperature control modules, the capping module and the magnetic field module, and controls the transport module to move the centrifuge tube carrying module, so that a centrifuge tube contained in the centrifuge tube carrying module makes a reaction in the temperature control modules, and the magnetic field module or the capping module is provided to the centrifuge tube according to requirements, such that a specimen in the centrifuge tube can be automatically subjected to nucleic acid extraction, nucleic acid amplification, primer labeling, reverse transcription or a combination thereof, thereby reducing manual operation errors and increasing the ease of operation.

### FIELD OF THE INVENTION

The present invention relates to a nucleic acid detection device, in particular to an automated molecular operating system that can automatically carry out several cycles at different reaction temperatures for liquid specimens such as blood, saliva, urine and spinal fluid according to their required reaction temperatures to achieve nucleic acid extraction, nucleic acid amplification, primer labeling, reverse transcription or other required biological operating steps.

### BACKGROUND OF THE INVENTION

With the development of modern medical technology and clinical needs, many diagnostic methods or judgments gradually need quantitative analysis for target genes. In particular, a technology of polymerase chain reaction (PCR) for a specimen can greatly increase the number of nucleic acid detection targets within a relatively short period of time, so that a small number of nucleic acid detection targets in the specimen can be increased to a detectable number, which is conducive to the subsequent detection of the presence or absence of a nucleic acid detection target to be detected, and even the determination of its number. Therefore, the technology of polymerase chain reaction has been widely used in medical and biological experimental detection.

However, the process of polymerase chain reaction requires multiple cycles at different temperatures, and the number of nucleic acid detection targets in the specimen is increased through each cycle at different temperatures. Therefore, when the specimen undergoes a polymerase chain reaction, each cycle roughly refers to denaturing a DNA template of the specimen by heating, then cooling for primer anneal, and then heating up to DNA synthesis in the specimen, so that the number of nucleic acids in a target in the specimen can be increased, and then enough nucleic acids can be obtained for detection after multiple cycles. However, most of the above procedures are carried out in a manual mode. Therefore, heating, cooling and other procedures of the specimen need to rely on personnel to pick up or move, which may cause the inconsistency in the temperature change of the specimen, or increase the man-hours of manually waiting for the specimen to react. In addition, when a user performs nucleic acid extraction, nucleic acid amplification or primer labeling on the specimen in a single-function device, both damages due to poor temperature control and unnecessary errors in the manual operation process are caused because the specimen needs to be transferred between devices with different functions. In view of this, the inventors hereby devote themselves to research and combine what they have learned. In view of the above problems, an automated method that can be used to carry out nucleic acid extraction, nucleic acid amplification, primer labeling or a combination thereof on the specimen in the same system environment according to set procedures and requirements is proposed to overcome the conventional lack.

### SUMMARY OF THE INVENTION

A main object of the invention is to provide an automated molecular operating system which can perform nucleic acid extraction, nucleic acid amplification, primer labeling, reverse transcription, or a combination thereof on a liquid specimen such as blood, saliva, urine, and spinal fluid in an automated way in the same system environment.

In order to achieve the above object, the invention provides an automated molecular operating system, for performing nucleic acid extraction, nucleic acid amplification, primer labeling or reverse transcription on a specimen contained in a centrifuge tube. The specimen is one of blood, saliva, urine, and spinal fluid. The automated molecular operating system includes at least one centrifuge tube carrying module, a transport module, a plurality of temperature control modules, a capping module, a magnetic field module, and an automated processing module. Each of the at least one centrifuge tube carrying module is provided with a plurality of first accommodating holes, at least one first buckling hole and at least one second buckling hole. The first accommodating holes are located between the first buckling hole and the second buckling hole. The transport module is provided with a first slide rail unit and a clamping drive unit assembled on the first slide rail unit, and the clamping drive unit clamps the at least one centrifuge tube carrying module to move in an axial direction of the first slide rail unit. Each of the plurality of temperature control modules is provided with a plurality of second accommodating holes and a temperature control unit. The temperature control unit controls a temperature of the temperature control module to achieve a required reaction temperature or be operated in a temperature change mode. The capping module is assembled at one end of the first slide rail and provided with a cover body. The magnetic field module is assembled on a circumferential side of one of the plurality of temperature control modules. The automated processing module is electrically connected to the transport module, the temperature control module, the capping module, and the magnetic field module. The automated processing module is configured to control a temperature of the temperature control unit of each of the plurality of temperature control modules to reach a set reaction temperature or controls the temperature control unit to be operated in a temperature change mode, and the automated processing module controls the clamping drive unit of the transport module to clamp the at least one the centrifuge tube carrying modules, so that the centrifuge tube carrying module is moved along the first slide rail unit, and the at least one centrifuge tube carrying module is selectively moved to the temperature control module with the magnetic field module for nucleic acid extraction, or the at least one centrifuge tube carrying module is selectively moved to and placed in the plurality of temperature control modules with different temperatures sequentially for nucleic acid amplification, or the at least one centrifuge tube carrying module is selectively moved to and placed in one of the plurality of temperature control modules filled with a reagent containing primers for primer labeling. When the clamping drive unit places the at least one centrifuge tube carrying module in one of the plurality of temperature control modules, the plurality of first accommodating hole are located correspondingly to the plurality of second accommodating hole.

In some embodiments, the clamping drive unit is further provided with a first body, a first driver, a second body, a second slide rail unit, a first support arm, a second support arm, a first clamp, a second clamp and a second driver. The first body is assembled at one end of the first driver and is located on the first slide rail unit, and the first driver is disposed on the circumferential side of the first slide rail unit. The second body is assembled above the first body, and the second slide rail unit is disposed on a side surface of the second body. The first support arm and the second support arm are slidably disposed on two opposite sides of the second slide rail unit respectively. One end of the first support arm is assembled on the first clamp, and an other end of the first support arm is assembled at one end of the second driver. One end of the second support arm is assembled on the second clamp, and the other end of the second support arm is assembled at the other end of the second driver. The second driver is assembled on the second body. The first driver controls the second body to move in an axial direction of the first driver to adjust a first distance between the second body and the first body, and the second driver adjusts a second distance between the first clamp and the second clamp.

In some embodiments, the second driver is provided with a rotating drive block, a first connecting rod and a second connecting rod. One end of the first connecting rod is connected to the first support arm and moves in an axial direction of the second slide rail unit, and an other end of the first connecting rod is pivoted to one end of the rotating drive block. One end of the second connecting rod is connected to the second support arm and moves in the axial direction of the second slide rail unit, and an other end of the second connecting rod is pivoted to an other end of the rotating drive block.

In some embodiments, a first locking groove is formed in an other end of the first clamp opposite to the end where the first support arm is assembled, and a second locking groove is formed in an other end of the second clamp opposite to the end where the second support arm is assembled. After the clamping drive unit clamps the at least one centrifuge tube carrying module, the rotating drive block of the second driver shortens the second distance, and the first locking groove is limited and locked in the first buckling hole of the at least one centrifuge tube carrying module, and the second locking groove is limited and locked in the second buckling hole of the at least one centrifuge tube carrying module.

In some embodiments, the capping module is provided with a third driver and at least one auxiliary rod. The third driver is disposed on the circumferential side of the first slide rail unit, and one end of the third driver is assembled on the cover body. The auxiliary rod is adjacent to the third driver and disposed with the cover body at one end thereof.

In some embodiments, the magnetic field module is provided with a fourth driver, at least one telescopic rod and a magnetic field generator. The fourth driver is assembled on the circumferential side of the first slide rail unit. One end of the telescopic rod is assembled on the fourth driver, and an other end of the telescopic rod and the magnetic field generator are assembled together. The fourth driver controls the length of the telescopic rod and adjusts a distance between the magnetic field generator and the temperature control module.

In some embodiments, the automated processing module controls the clamping drive unit to clamp the at least one centrifuge tube carrying module first to the temperature control module with the magnetic field module for nucleic acid amplification, and then place the at least one centrifuge tube carrying module in one of the plurality of temperature control modules filled with the reagent containing primers for primer labeling; or clamp the at least one centrifuge tube carrying module first to the temperature control module with the magnetic field module for nucleic acid extraction, and then clamp and place the centrifuge tube carrying module in the plurality of temperature control modules with different temperatures sequentially for nucleic acid amplification; or clamp the centrifuge tube carrying module first to the temperature control module with the magnetic field module for nucleic acid extraction, and then clamp and place the at least one centrifuge tube carrying module in the plurality of temperature control modules with different temperatures sequentially for nucleic acid amplification, and then place the at least one centrifuge tube carrying module in one of the plurality of temperature control modules filled with the reagent containing primers for primer labeling.

Accordingly, the automated molecular operating system of the present invention has the following beneficial effects compared with the prior art:

The automated molecular operating system of the present invention utilizes the automated processing module to control the clamping drive unit of the transport module to clamp one of the centrifuge tube carrying module to move along the axial direction of the first slide rail unit in a set mode. So that the specimens in the centrifuge tubes placed in the first receiving holes of the centrifuge tube carrying module can automatically move to the temperature control module of the magnetic field module performs nucleic acid extraction, or is placed in the temperature control modules with different temperatures in turn to perform nucleic acid amplification, or is placed in one of the temperature control modules for primer labeling, or perform nucleic acid extraction in the temperature control module with the magnetic field module, and then choose whether placing the specimens in the temperature control modules with different temperatures in turn to perform nucleic acid amplification according to the requires, or after completing nucleic acid amplification, then choose whether placing the specimens in one of the temperature control modules for primer labeling. In this way, the user can adjust the temperature control module according to needs in the automated molecular operating system. Nucleic acid extraction, nucleic acid amplification, or primer labeling can be performed individually in an automated manner, or nucleic acid amplification, primer labeling, or a combination thereof can be performed after nucleic acid extraction according to the needs. Therefore, the specimens can be transferred and reacted in an automated way in the same system environment, which not only reduces the labor cost, but also avoids manual operation errors and increases the convenience of operation.

### BRIEF DESCRIPTION OF THE DRAWINGS

FIG. 1 is a schematic diagram of an automated molecular operating system of the present invention;

FIG. 2 is a schematic diagram of the automated molecular operating system of the present invention from another perspective;

FIG. 3 is an exploded view of the automated molecular operating system of the present invention;

FIGS. 4-1 and 4-2 are schematic diagrams showing the operation of a rotation drive block of the automated molecular operating system of the present invention to control a clamping drive unit to clamp a centrifuge tube carrying module;

FIGS. 4-3, 4-4, and 4-5 are schematic diagrams showing the operation of the clamping drive unit of the automated molecular operating system of the present invention to move the centrifuge tube carrying module to a temperature control module; and

FIG. 5 is a block diagram showing the operation of the automated molecular operating system of the present invention.

### DETAILED DESCRIPTION OF THE PREFERRED EMBODIMENTS

Several embodiments are listed for technical features and modes of operation in the present application in conjunction with accompanying drawings for review and reference. Furthermore, the accompanying drawings in the present invention are not necessary to scale for the sake of description, and the scales in the accompanying drawings are not intended to limit the protection scope of the present invention.

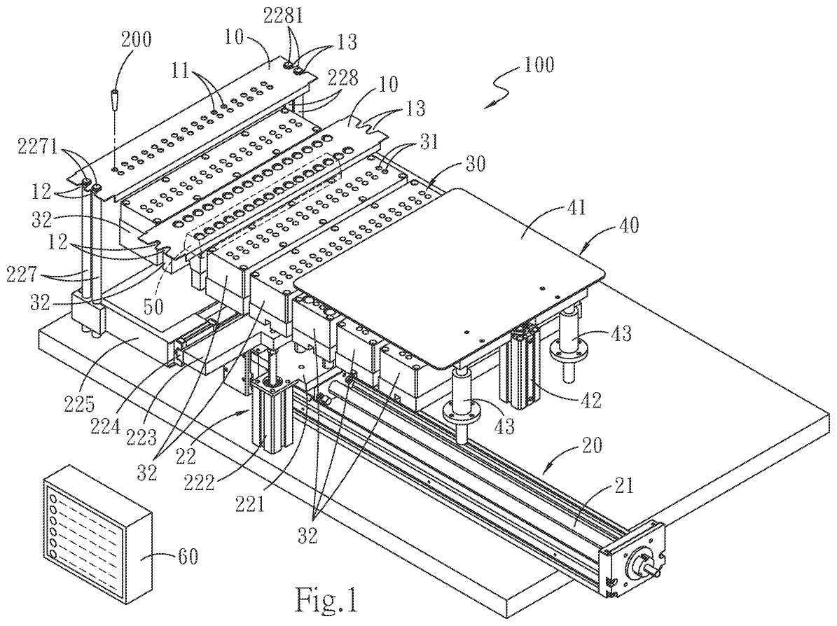

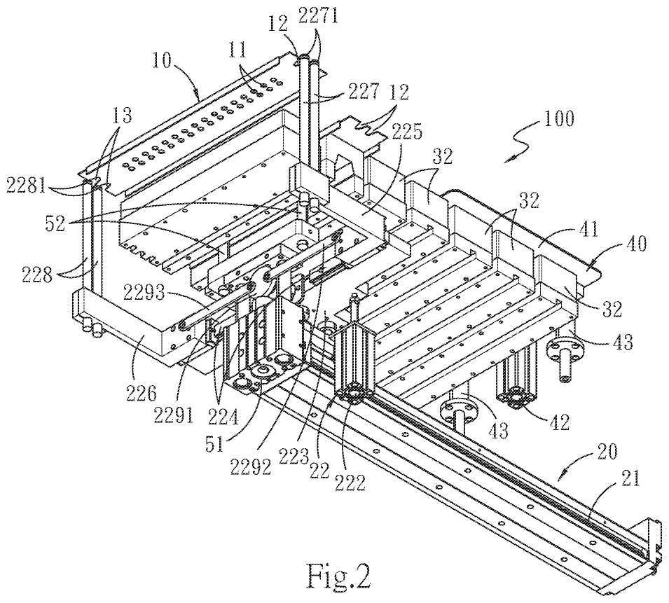

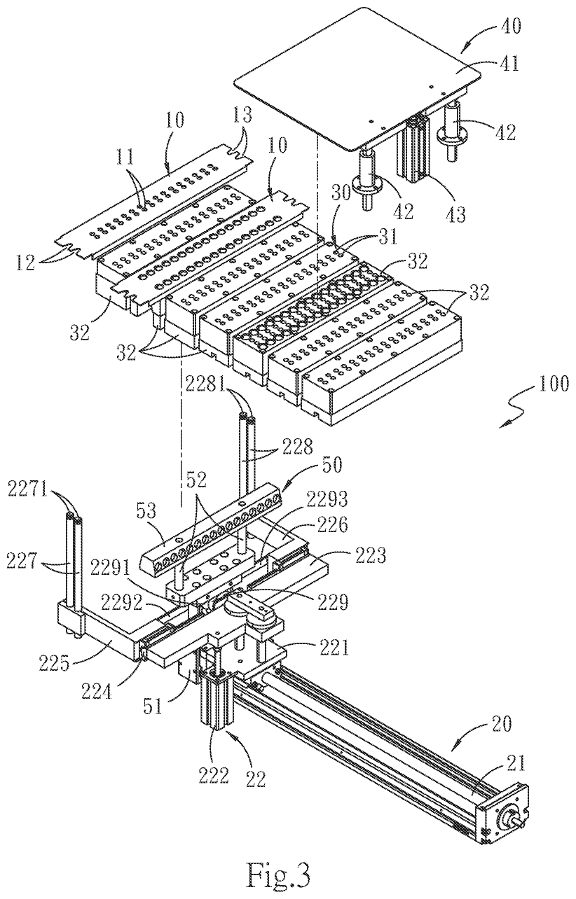

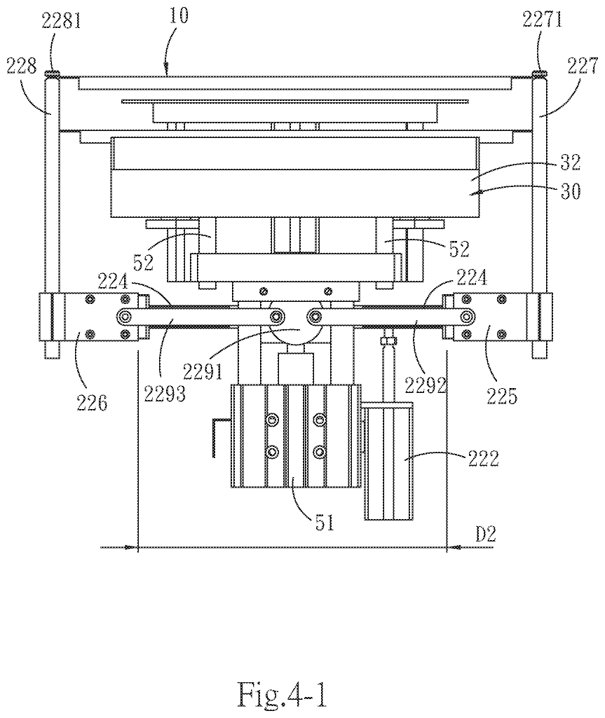

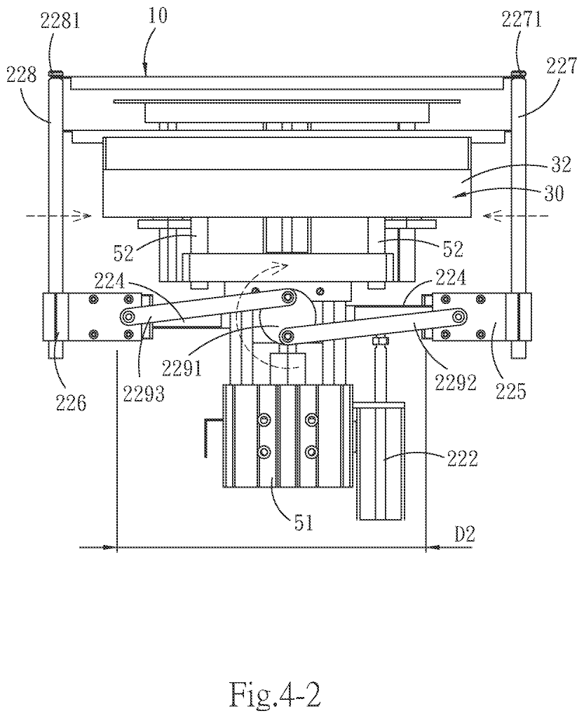

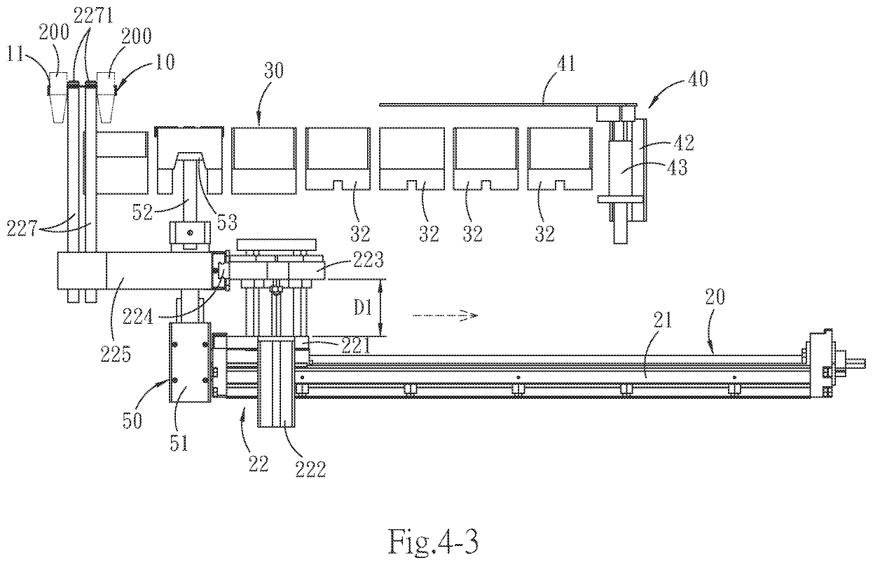

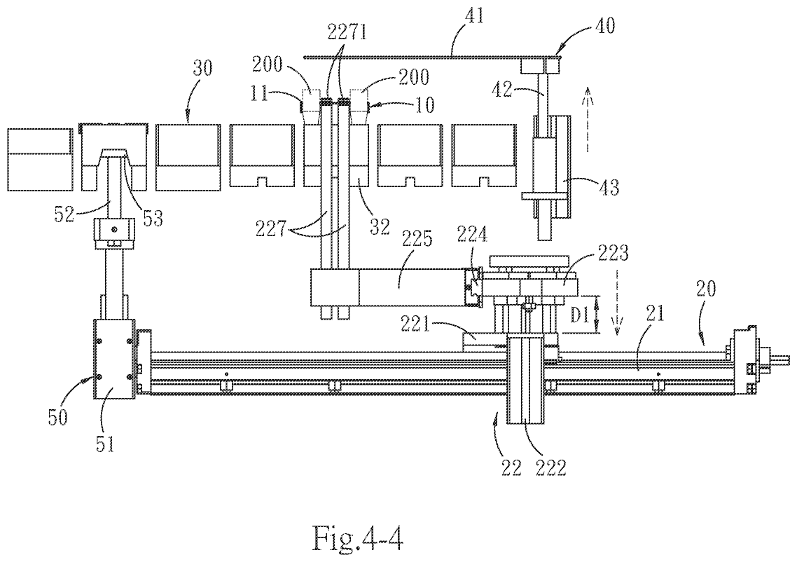

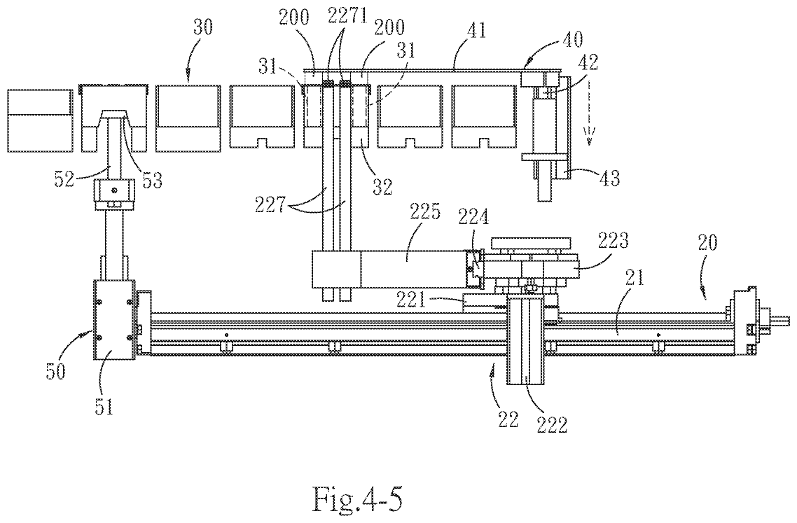

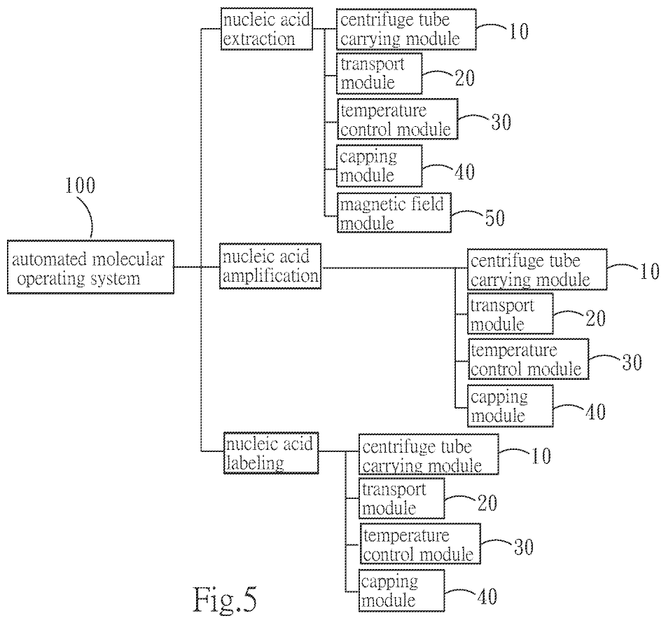

Regarding the content of the present invention, please refer to an automated molecular operating system 100 provided by the present invention shown in FIGS. 1, 2, 3, 4-1, 4-2, 4-3, 4-4, 4-5, and 5, which is used to perform nucleic acid extraction, nucleic acid amplification, primer labeling, reverse transcription or other biological operations on a specimen contained in a centrifuge tube 200. The automated molecular operating system 100 includes a plurality of centrifuge tube carrying modules 10, a transport module 20, a plurality of temperature control modules 30, a capping module 40, a magnetic field module 50 and an automated processing module 60. Therefore, referring to FIG. 5 first, the specimen in the centrifuge tube 200 contains one of liquid specimens such as blood, saliva, urine or spinal fluid. The automated molecular operating system 100 can be set up to perform nucleic acid extraction, nucleic acid amplification, primer labeling, reverse transcription or other biological operations on the specimen according to the needs thereof. The automated molecular operating system 100 is configured to select the corresponding modules and processes from the transport module 20, the temperature control modules 30, the capping module 40 and the magnetic field module 50 for the centrifuge tube carrying modules 10 according to a selected mode.

Each of the centrifuge tube carrying modules 10 is provided with a plurality of first accommodating holes 11, two first buckling holes 12 and two second buckling holes 13. Referring to FIGS. 1, 2, 3, 4-1, 4-2, 4-3, 4-4, and 4-5 again, the first accommodating holes 11 are located between the first buckling holes 12 and the second buckling holes 13.

The transport module 20 is provided with a first slide rail unit 21 and a clamping drive unit 22, wherein the clamping drive unit 22 is assembled on the first slide rail unit 21, and the clamping drive unit 22 clamps one of the centrifuge tube carrying modules 10 to move in an axial direction of the first slide rail unit 21. The clamping drive unit 22 is further provided with a first body 221, a first driver 222, a second body 223, a second slide rail unit 224, a first support arm 225, a second support arm 226, a first clamp 227, a second clamp 228 and a second driver 229. The first body 221 is assembled at one end of the first driver 222 and is located on the first slide rail unit 21, and the first driver 222 is disposed on a circumferential side of the first slide rail unit 21. The second body 223 is assembled above the first body 221, and the second slide rail unit 224 is disposed on a side surface of the second body 223. The first support arm 225 and the second support arm 226 are slidably disposed on two opposite sides of the second slide rail unit 224 respectively. One end of the first support arm 225 is assembled on the first clamp 227, and the other end of the first support arm 225 is assembled at one end of the second driver 229. One end of the second support arm 226 is assembled on the second clamp 228, and the other end of the second support arm 226 is assembled at the other end of the second driver 229. The second driver 229 is assembled on the second body 223 and is provided with a rotating drive block 2291, a first connecting rod 2292 and a second connecting rod 2293. One end of the first connecting rod 2292 is connected to the first support arm 225 and moves in an axial direction of the second slide rail unit 224, and the other end of the first connecting rod 2292 is pivoted to one end of the rotating drive block 2291. One end of the second connecting rod 2293 is connected to the second support arm 226 and moves in the axial direction of the second slide rail unit 224, and the other end of the second connecting rod 2293 is pivoted to the other end of the rotating drive block 2291. In addition, a first locking groove 2271 is formed in the other end of the first clamp 227 opposite to the end where the first support arm 225 is assembled, and a second locking groove 2281 is formed in the other end of the second clamp 228 opposite to the end where the second support arm 226 is assembled. The first driver 222 controls the second body 223 to move in an axial direction of the first driver 222, and causes the first driver 222 to control a distance D1 between the second body 223 and the first body 221. The second driver 229 controls a second distance D2 between the first clamp 227 and the second clamp 228. Further, after the clamping drive unit 22 clamps one of the centrifuge tube carrying modules 10, the rotating drive block 2291 of the second driver 229 shortens the second distance D2. The first locking groove 2271 is limited and locked in the first buckling hole 12 of the centrifuge tube carrying module 10, and the second locking groove 2281 is limited and locked in the second buckling hole 13 of the centrifuge tube carrying module 10.

Each of the temperature control modules 30 is provided with a plurality of second accommodating holes 31 and a temperature control unit 32. The temperature control unit 32 allows the temperature control module 30 to reach a required reaction temperature or be controlled in a temperature change mode. The temperature control module 30 in this embodiment is provided with four groups of temperature control modules 30 where temperature control units 32 function as heaters so that the four groups of temperature control modules 30 have a temperature higher than the room temperature; a group of temperature control modules 30 where temperature control units 32 function as coolers so that this group of temperature control modules 30 has a temperature equivalent to 0° C.; and two further groups of temperature control modules 30 where temperature control units 32 function as thermostats so that these two groups of temperature control modules 30 have a temperature equivalent to room temperature, but are not limited thereto;

The capping module 40 is assembled at one end of the first slide rail unit 21. The capping module 40 is provided with a cover body 41, a third driver 42 and two auxiliary rods 43. The third driver 42 is disposed on the circumferential side of the first slide rail unit 21, and one end of the third driver 42 is assembled on the cover body 41. The auxiliary rods 43 are adjacent to the third driver 42, and disposed with their one ends to the cover body 41 respectively. When the auxiliary rods 43 raise and lower the cover body 41 by means of the third driver 42, the auxiliary rods 43 may assist the cover body 41 to be raised and lowered, extending the operating life of the third driver 42. The cover body 41 is mainly used for covering the temperature control modules 30 where temperature control units 32 function as the heaters for the temperature control modules 30 to achieve a heating purpose, a liquid in the centrifuge tubes 200 is protected against evaporation, but is not limited thereto.

The magnetic field module 50 is assembled on the circumferential side of one of the temperature control modules 30. Further, the magnetic field module 50 is provided with a fourth driver 51, two telescopic rods 52 and a magnetic field generator 53. The fourth driver 51 is assembled on the circumferential side of the first slide rail unit 21. One end of each telescopic rod 52 is assembled on the fourth driver 51, and the other end of the telescopic rod 52 and the magnetic field generator 53 are assembled together. The fourth driver 51 controls the length of the telescopic rod 52 and adjusts a distance between the magnetic field generator 53 and the temperature control module 30. Further, the magnetic field generator 53 of the magnetic field module 50 in this embodiment is disposed on the lower side of the temperature control module 30. The magnetic field generator 53 may also be disposed on the circumferential side of the temperature control module 30, and specific antibody magnetic beads (not shown) may be customized to provide the magnetic field generator 53 to purify specific proteins for extraction or perform reverse transcription on specific RNA.

The automated processing module 60 is electrically connected to the transport module 20, the temperature control module 30, the capping module 40 and the magnetic field module 50.

The automated processing module 60 controls the temperature control units 32 of the temperature control modules 30. The temperature control modules 30 cause the temperature control units 32 to reach a set reaction temperature or temperature change mode respectively, and control the clamping drive unit 22 of the transport module 20 to clamp one of the centrifuge tube carrying modules 10, so that the centrifuge tube carrying module 10 moves along the first slide rail unit 21, and the centrifuge tube carrying module 10 selects to move to the temperature control module 30 with the magnetic field module 50 for nucleic acid extraction. The nucleic acid extraction is mainly carried out by extracting deoxyribonucleic acid (DNA), ribonucleic acid (RNA) or molecular marker; or clamping the centrifuge tube carrying module 10 and placing it in turn in the temperature control modules 30 with different temperatures for nucleic acid amplification; or clamping the centrifuge tube carrying module 10 and place it in one of the temperature control modules 30 to be filled with a reagent containing primers and labeling all the primers on the specimen for primer labeling; or clamping the centrifuge tube carrying module 10 to the temperature control module 30 with the magnetic field module 50 for nucleic acid extraction; or then filling in one of the temperature control modules 30 with the reagent containing the primers and labeling all the primers on the specimen for primer labeling; or clamping the centrifuge tube carrying module 10 first to the temperature control module 30 with the magnetic field module 50 for nucleic acid extraction, and then clamping the centrifuge tube carrying module 10 and placing it in turn in the temperature control modules 30 with different temperatures for nucleic acid amplification; or clamping the centrifuge tube carrying module 10 first to the temperature control module 30 with the magnetic field module 50 for nucleic acid extraction, and then clamping the centrifuge tube carrying module 10 and place it in turn in the temperature control modules 30 with different temperatures for high temperature denaturation. The primers are annealed at low temperature to extend or elongation a sequence to achieve an amplification effect for nucleic acid amplification. Next, one of the temperature control modules 30 is filled with the reagent containing primers and all the primers are labeled on the specimen for primer labeling. The temperature control unit 32 allows the centrifuge tube 200 disposed into the second accommodating hole 31 of the temperature control module 30 to reach a set reaction temperature or temperature change mode respectively. In addition, when the clamping drive unit 22 places one of the centrifuge tube carrying modules 10 in the corresponding temperature control module 30, the first accommodating hole 11 is opposite to the second accommodating hole 31. Further, reverse transcription may also be performed after the above nucleic acid extraction. The reverse transcription is performed by first transcribing DNA of the specimen to obtain mRNA (messenger RNA), and then reversely transcripting mRNA into cDNA (complementary DNA) by reacting mRNA with reverse transcriptase through Oligo (dT) (polythymine, T-repeat oligonucleotide), while Oligo (dT) is a nucleotide chain composed of thymine, allowing it to produce only required cDNA.

Further, as shown in FIGS. 3, 4-1, 4-2, 4-3, 4-4, and 4-5, the user places the specimen to be extracted in the centrifuge tube 200, and places the centrifuge tube 200 in which the specimen has been placed and which has undergone the same reaction temperature or temperature change procedure in the first accommodating hole 11 of the centrifuge tube carrying module 10. The required reagent may be added to the centrifuge tube 200 to react with the specimen. The centrifuge tube carrying module 10 whose centrifuge tube 200 has been placed in the first accommodating hole 11 may first be placed in the temperature control module 30, and then the user sets the automated processing module 60 according to demands. Referring to FIG. 1, the temperature control modules 30 in this embodiment, from left to right, are one cooler, two thermostats, and four heater. The temperature control units 32 of the temperature control module 30 at the left side has function as coolers. Two of temperature control modules 30 where temperature control units 32 function as thermostats and are kept at room temperature, one of which is provided with the magnetic field module 50. and four of temperature control modules 30 where temperature control units 32 function as heaters and which have different temperatures above room temperature, three groups of which may be covered by the cover body 41 of the capping module 40. Referring first to FIG. 4-1 to FIG. 5, the automated processing module 60 on the one hand enables the temperature control module 30 to control the corresponding temperature control unit 32 to reach a default reaction temperature or a temperature change mode to be carried out, and on the other hand, controls the second driver 229 of the clamping drive unit 22. The second driver 229 controls the first support arm 225 and the second support arm 226 to move in the axial direction of the second slide rail unit 224, so that the second distance D2 between the first support arm 225 and the second support arm 226 is gradually shortened, and the first locking groove 2271 of the first clamp 227 is locked in the first buckling hole 12 of the centrifuge tube carrying module 10 to be moved. The second locking groove 2281 of the second clamp 228 is locked in the second buckling hole 13 of the centrifuge tube carrying module 10 to be moved. That is, the second driver 229 will control the rotating drive block 2291 to rotate clockwise or counterclockwise, so that the rotating drive block 2291 drives the first connecting rod 2292 and the second connecting rod 2293 pivoted at both ends. In this way, the first connecting rod 2292 will drive the first supporting arm 225 connected to the other end to move in the axial direction of the second slide rail unit 224, and the second connecting rod 2293 will also drive the second supporting arm 226 connected to the other end to move in the axial direction of the second slide rail unit 224. By changing the length of the second distance D2, the first clamp 227 and the second clamp 228 of the clamping drive unit 22 may clamp one of the centrifuge tube carrying modules 10, and allows the centrifuge tube carrying module 10 to move in the axial direction of the first slide rail unit 21.

In addition, referring to FIG. 4-3 to FIG. 4-5 again, when the first clamp 227 and the second clamp 228 of the clamping drive unit 22 clamp one of the centrifuge tube carrying modules 10, the automated processing module 60 will control the clamping drive unit 22 so that the centrifuge tube carrying module 10 moves in the axial direction of the first slide rail unit 21. When the centrifuge tube carrying module 10 moves to the corresponding one of the temperature control modules 30, the automated processing module 60 will control the first driver 222, and the first driver 222 will allow the second body 223 to move towards the first body 221, that is, the first distance D1 between the second body 223 and the first body 221 will be shortened, and the centrifuge tubes 200 in the first accommodating holes 11 will be placed into the corresponding second accommodating holes 31 one by one. In addition, after the automated processing module 60 controls the temperature control unit 32 so that the temperature control module 30 reaches a predetermined reaction temperature or temperature change mode, the automated processing module 60 will clamp the corresponding centrifuge tube carrying module 10 according to the settings, so that the specimen in the centrifuge tube 200 of the centrifuge tube carrying module 10 can only move to the temperature control module 30 with the magnetic field module 50, and the centrifuge tubes 200 are placed in the corresponding second accommodating holes 31 one by one. The automated processing module 60 controls the fourth driver 51 of the magnetic field module 50, so that the telescopic rod 52 locates the magnetic field generator 53 on the circumferential side of the centrifuge tube 200 to carry out magnetic bead extraction of the specimen in the centrifuge tube 200. The magnetic field generator 53 may be disposed below the temperature control module 30, or disposed around the temperature control module 30. The magnetic field generator 53 in this embodiment is disposed below the temperature control module 30, but is not limited thereto.

Further, when the user only needs to carry out nucleic acid amplification, the automated processing module 60 will clamp the corresponding one of the centrifuge tube carrying modules 10 according to the settings, so that the specimen in the centrifuge tube 200 of the centrifuge tube carrying module 10 is placed in the second accommodating hole 31 of the temperature control module 30 at the corresponding reaction temperature, and the specimen in the centrifuge tube 200 can reach a required reaction temperature and then reach a required number of nucleic acids. Furthermore, if the user wants to carry out primer labeling, the automated processing module 60 will clamp the corresponding one of the centrifuge tube carrying modules 10 according to the settings, place it in the second accommodating hole 31 of the temperature control module 30, and then fill a reagent containing primers in the centrifuge tube 200 for primer labeling. In addition, the user may also input instructions into this automated processing module 60 according to needs, so that this automated molecular operating system 100 can not only carry out nucleic acid extraction, nucleic acid amplification, primer labeling or reverse transcription on blood, saliva, urine, spinal fluid or other liquid specimens alone, but also can carry out primer labeling after nucleic acid extraction, or carry out nucleic acid amplification after nucleic acid extraction, and then carry out different biological operation procedures such as primer labeling. Then, in order to avoid the reagent added in the centrifuge tube 200 from escaping due to high temperature in the heating process, the capping module 40 will be disposed at the temperature control module 30 where the temperature control unit 32 functions as a heater, so that when the centrifuge tube placed in the centrifuge tube carrying module 10 is placed in the temperature control module 30 where the temperature control unit 32 functions as the heater, the automated processing module 60 controls the third driver 42, and the third driver 42 causes the cover body 41 to cover the centrifuge tube 200 of the centrifuge tube carrying module 10. When the centrifuge tube is to be moved to other temperature control modules 30 or to carry out subsequent extraction actions, the cover body 41 is raised again. Therefore, the automated molecular operating system 100 automatically moves the centrifuge tube carrying module 10 by the automated processing module 60 according to a set procedure, so that the centrifuge tube 200 reaches a required reaction temperature or temperature change mode in the corresponding temperature control module 30, and then automatically completes the required temperature change and the number of cycles, so as to achieve the purposes of nucleic acid extraction, nucleic acid amplification, primer labeling, reverse transcription or a combination thereof, thereby reducing manual operation errors and increasing the convenience.

In summary, the automated molecular operating system 100 of the present invention mainly controls the clamping drive unit 22 of the transport module 20 by the automated processing module 60, so that the clamping drive unit 22 clamps one of the centrifuge tube carrying modules 10 so that it can move in the axial direction of the first slide rail unit 21 according to a set mode. The specimen in the centrifuge tube 200 placed in the first accommodating hole 11 of the centrifuge tube carrying module 10 can be automatically moved to the temperature control module 30 with the magnetic field module 50 for nucleic acid extraction, or placed in the temperature control modules 30 with different temperatures in turn to carry out nucleic acid amplification, or placed in one of the temperature control modules 30 and filled with the reagent containing the primers to carry out primer labeling; or after nucleic acid extraction in the temperature control module 30 with the magnetic field module 50, whether the specimen needs to be placed in the temperature control modules 30 with different temperatures in turn to carry out nucleic acid amplification is selected according to needs; or after the nucleic acid amplification is completed, whether the specimen is placed in one of the temperature control modules 30 and filled with the reagent containing the primers to carry out biological procedures such as primer labeling or reverse transcription is determined. In this way, the user is allowed to carry out nucleic acid extraction, nucleic acid amplification, primer labeling, reverse transcription or the above combination in an automated manner according to the needs in the automated molecular operating system 100. That is, nucleic acid amplification or the combination of nucleic acid amplification and primer labeling is performed according to the needs after nucleic acid extraction. Therefore, the specimens can be transferred and reacted in an automated way in the same system environment, which not only reduces the labor cost, but also avoids manual operation errors and increases the convenience of operation.

What is claimed is:

1. An automated molecular operating system, for performing nucleic acid extraction, nucleic acid amplification, primer labeling or reverse transcription on a specimen contained in a centrifuge tube, comprising:
at least one centrifuge tube carrying module, each centrifuge tube carrying module provided with a plurality of first accommodating holes, at least one first buckling hole and at least one second buckling hole, the first accommodating holes located between the first buckling hole and the second buckling hole;
a transport module, provided with a first slide rail unit and a clamping drive unit assembled on the first slide rail unit, the clamping drive unit clamping the at least one centrifuge tube carrying module to move in an axial direction of the first slide rail unit;
a plurality of temperature control modules, each of the plurality of temperature control modules provided with a plurality of second accommodating holes and a temperature control unit, the temperature control unit controlling a temperature of the temperature control module to achieve a required reaction temperature or be operated in a temperature change mode;
a capping module, assembled at one end of the first slide rail and provided with a cover body;
a magnetic field module, assembled on a circumferential side of one of the plurality of temperature control modules; and
an automated processing module, electrically connected to the transport module, the temperature control module, the capping module and the magnetic field module;
wherein the automated processing module is configured to control a temperature of the temperature control unit of each of the plurality of temperature control modules to reach a set reaction temperature or controls the temperature control unit to be operated in a temperature change mode, and the automated processing module controls the clamping drive unit of the transport module to clamp the at least one the centrifuge tube carrying modules, so that the centrifuge tube carrying module is moved along the first slide rail unit, and the at least one centrifuge tube carrying module is selectively moved to the temperature control module with the magnetic field module for nucleic acid extraction, or the at least one centrifuge tube carrying module is selectively moved to and placed in the plurality of temperature control modules with different temperatures sequentially for nucleic acid amplification, or the at least one centrifuge tube carrying module is selectively moved to and placed in one of the plurality of temperature control modules filled with a reagent containing primers for primer labeling; and
wherein when the clamping drive unit places the at least one centrifuge tube carrying module in one of the plurality of temperature control modules, the plurality of first accommodating hole are located correspondingly to the plurality of second accommodating hole.

2. The automated molecular operating system according to claim 1, wherein the clamping drive unit is further provided with a first body, a first driver, a second body, a second slide rail unit, a first support arm, a second support arm, a first clamp, a second clamp and a second driver, and wherein the first body is assembled at one end of the first driver and is located on the first slide rail unit, and the first driver is disposed on the circumferential side of the first slide rail unit, and wherein the second body is assembled above the first body, and the second slide rail unit is disposed on a side surface of the second body, and wherein the first support arm and the second support arm are slidably disposed on two opposite sides of the second slide rail unit respectively, and wherein one end of the first support arm is assembled on the first clamp, and an other end of the first support arm is assembled at one end of the second driver, and wherein one end of the second support arm is assembled on the second clamp, and the other end of the second support arm is assembled at the other end of the second driver, and wherein the second driver is assembled on the second body, and wherein the first driver is configured to control the second body to move in an axial direction of the first driver to adjust a first distance between the second body and the first body, and the second driver is configured to adjusts a second distance between the first clamp and the second clamp.

3. The automated molecular operating system according to claim 2, wherein the second driver is provided with a rotating drive block, a first connecting rod and a second connecting rod, and wherein one end of the first connecting rod is connected to the first support arm and moves in an axial direction of the second slide rail unit, and an other end of the first connecting rod is pivoted to one end of the rotating drive block, and wherein one end of the second connecting rod is connected to the second support arm and moves in the axial direction of the second slide rail unit, and an other end of the second connecting rod is pivoted to an other end of the rotating drive block.

4. The automated molecular operating system according to claim 2, wherein a first locking groove is formed in an other end of the first clamp opposite to the end where the first support arm is assembled, and a second locking groove is formed in an other end of the second clamp opposite to the end where the second support arm is assembled, and wherein after the clamping drive unit clamps the at least one centrifuge tube carrying module, a rotating drive block of the second driver shortens the second distance, and the first locking groove is limited and locked in the first buckling hole of the at least one centrifuge tube carrying module, and the second locking groove is limited and locked in the second buckling hole of the at least one centrifuge tube carrying module.

5. The automated molecular operating system according to claim 1, wherein the capping module is provided with a third driver and at least one auxiliary rod, and wherein the third driver is disposed on the circumferential side of the first slide rail unit, and one end of the third driver is assembled on the cover body, and wherein the auxiliary rod is adjacent to the third driver and disposed with the cover body at one end thereof.

6. The automated molecular operating system according to claim 1, wherein the magnetic field module is provided with a fourth driver, at least one telescopic rod and a magnetic field generator, and wherein the fourth driver is assembled on the circumferential side of the first slide rail unit, and wherein one end of the telescopic rod is assembled on the fourth driver, and an other end of the telescopic rod and the magnetic field generator are assembled together, and wherein the fourth driver controls a length of the telescopic rod and adjusts a distance between the magnetic field generator and the temperature control module.

7. The automated molecular operating system according to claim 1, wherein the automated processing module is configured to control the clamping drive unit to clamp the at least one centrifuge tube carrying module first to the temperature control module with the magnetic field module for nucleic acid amplification, and then place the at least one centrifuge tube carrying module in one of the plurality of temperature control modules filled with the reagent containing primers for primer labeling; or clamp the at least one centrifuge tube carrying module first to the temperature control module with the magnetic field module for nucleic acid extraction, and then clamp and place the centrifuge tube carrying module in the plurality of temperature control modules with different temperatures sequentially for nucleic acid amplification; or clamp the centrifuge tube carrying module first to the temperature control module with the magnetic field module for nucleic acid extraction, and then clamp and place the at least one centrifuge tube carrying module in the plurality of temperature control modules with different temperatures sequentially for nucleic acid amplification, and then place the at least one centrifuge tube carrying module in one of the plurality of temperature control modules filled with the reagent containing primers for primer labeling.
